(19) United States
(12) Patent Application Publication
(10) Pub. No.: US 2012/0272971 A1
(43) Pub. Date: Nov. 1, 2012

(54) EXTENSION CONDOM

(76) Inventors: Mark Melnick, Orlando, FL (US); Marina Melnick, Orlando, FL (US)

(21) Appl. No.: 13/096,089

(22) Filed: Apr. 28, 2011

(51) Int. Cl. A61F 6/04

(52) U.S. Cl. 128/844

(57) ABSTRACT

An extension condom includes a first thin elastic tubular sheath having an open end and a closed end, a sperm containment cell, a second thin elastic tubular sheath connected to the closed end of the first tubular sheath, and an extension material configured to provide an appearance and feel of a longer penis.

### BACKGROUND

### Field of the Invention

The present invention relates generally to prophylactic and prosthetic devices, and more particularly to a male contraceptive device or condom having an improved shape for providing the appearance of a larger penis.

Contraceptives such as male condoms are extremely well known in the art and include U.S. Pat. No. 4,869,270, U.S. Pat. No. 4,281,648 and U.S. Pat. No. 7,673,632, among many others. Since the initial creation of these devices, many improvements have been made towards increasing the reliability/structural integrity of the condom itself, and by including additives such as spermicide to protect against pregnancy. Additionally, there have been attempts to increase the enjoyment to the wearers' partner by including pleasurable aspects such as ribs or ridges.

However, there remains a need for a condom capable of providing the appearance and feel of a longer penis, while still providing the safety and security of a traditional condom. To this end, the present invention differs from the foregoing prior art in a number of aspects, the manner by which will become more apparent in the description which follows, particularly when read in conjunction with the accompanying drawings.

### SUMMARY OF THE INVENTION

The present invention is directed to an extension condom. One embodiment of the present invention can include a first thin elastic tubular sheath having an open end and a closed end, a sperm containment cell, a second thin elastic tubular sheath connected to the closed end of the first tubular sheath, and an extension material configured to provide an appearance and feel of a longer penis.

Another embodiment of the present invention can include a memory extension material for allowing a user to customize the shape of the extension material.

### BRIEF DESCRIPTION OF THE DRAWINGS

Presently preferred embodiments are shown in the drawings. It should be appreciated, however, that the invention is not limited to the precise arrangements and instrumentalities shown.

FIG. 1 is a side illustration of an extension condom in a rolled configuration according to one embodiment of the present invention.

FIG. 2 is a perspective illustration of an extension condom in a rolled configuration according to one embodiment of the present invention.

FIG. 3 is a side illustration of an extension condom in an extended configuration according to one embodiment of the present invention.

FIG. 4 is a side illustration of an extension condom in an extended configuration according to another embodiment of the present invention.

### DETAILED DESCRIPTION OF THE INVENTION

While the specification concludes with claims defining the features of the invention that are regarded as novel, it is believed that the invention will be better understood from a consideration of the description in conjunction with the drawings. As required, detailed embodiments of the present invention are disclosed herein; however, it is to be understood that the disclosed embodiments are merely exemplary of the invention, which can be embodied in various forms. Therefore, specific structural and functional details disclosed herein are not to be interpreted as limiting, but merely as a basis for the claims and as a representative basis for teaching one skilled in the art to variously employ the inventive arrangements in virtually any appropriately detailed structure. Further, the terms and phrases used herein are not intended to be limiting but rather to provide an understandable description of the invention.

As described throughout this disclosure, a “user” is to be construed as the person wearing the condom, and the condom 10 itself can be manufactured to form a single integral object using an appropriately shaped mold (not illustrated) according to known manufacturing processes.

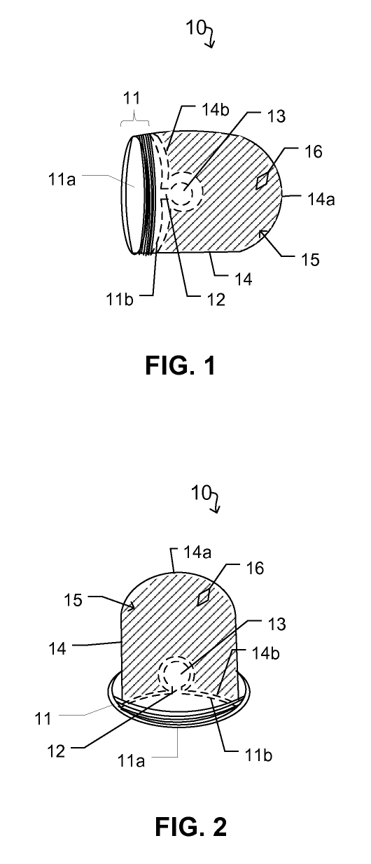

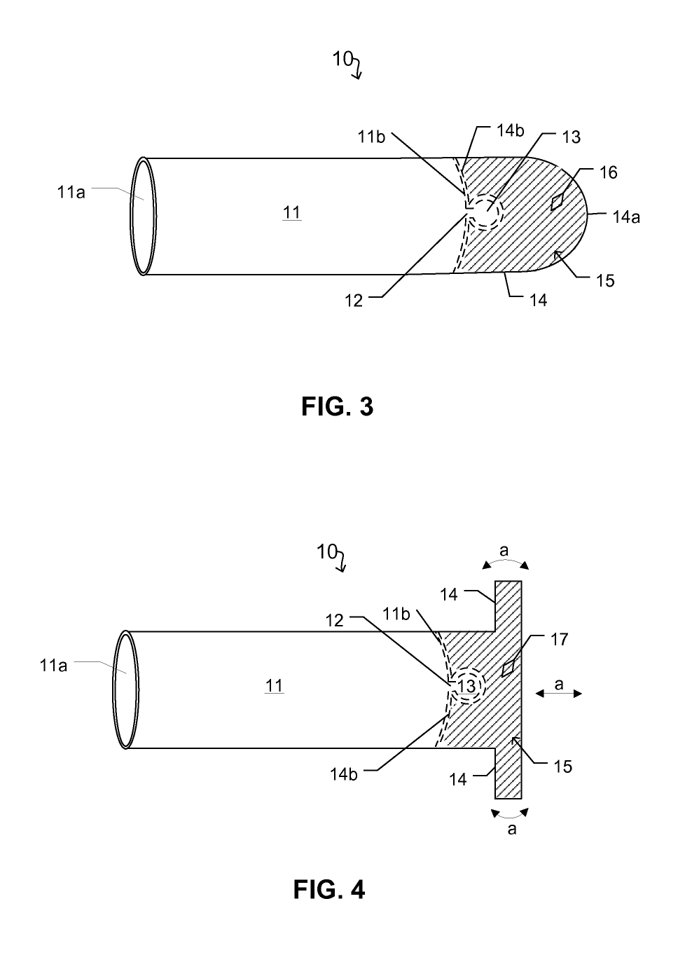

FIGS. 1-3 illustrate one embodiment of an extension condom 10 that is useful for understanding the inventive concepts disclosed herein. As shown, the condom 10 is in an extended configuration in FIG. 3, and in a rolled configuration in FIGS. 1 and 2 which would be suitable for packaging and commercial vending. The condom 10, according to one embodiment, is an elastic disposable male condom that includes a posterior section or sheath like body 11, and an anterior portion or sheath like body 14 having an extension material 16 positioned therein.

The posterior sheath 11 acts as a traditional condom for covering the penis of a user, and includes an interior and exterior surface having an open end 11a and a closed end 11b. As shown, a sperm containment cell 13 can be incorporated into the closed end of the posterior sheath 1 lb via a horizontal neck 12. The purpose and manner by which the neck 12 and sperm containment cell 13 are used is well known.

The anterior sheath 14 acts to position an extension material 16 onto the end of the posterior sheath 11, and includes an interior and exterior surface having a closed end 14a acting as the tip of the condom, and an open end 14b for connecting to the closed end of the posterior sheath 11b. When so positioned, the anterior sheath 14 forms a pocket 15 for securely containing the extension material 16.

In one preferred embodiment, the posterior sheath 11 can include a length of between ¼ of an inch to two inches and a girth approximating the inner diameter of the first and second sheaths, however other sizes are also contemplated. Moreover, it is also preferred that the posterior sheath 11 be integrally formed with the anterior sheath 14 according to processes known to those skilled in the art, so as to hide the extension material 16. Such a process will give the appearance of a normal condom when worn on the penis of a user. To this end, both the posterior and anterior sheaths can be constructed from traditional condom materials such as latex, polyurethane and lamb skin, for example, and can include any number of different colors, sizes and/or marking combinations.

The extension material 16 can be positioned within the pocket 15 so as to surround the sperm containment cell 13. The extension material 15 can act to provide the appearance and feel of a longer penis during intercourse. To this end, the extension material 16 can be constructed from any number of soft, semi-rigid and/or malleable materials such as, for example, soft rubber, foam, liquid, gel or formed latex, among many other materials having a weight and consistency suitable for this purpose. In one preferred embodiment, the extension material can be introduced into the pocket 15 prior to the marrying of the anterior and posterior sheaths, however other construction methodologies are also contemplated.

In operation, a user's penis can be placed into the sheath 11 until resting against the closed wall 11b, at which time the anterior sheath can be rolled back to cover the entirety of the penis in a traditional manner. When so positioned, the anterior sheath 14 containing the extension material 16 will extend outward in the same manner and direction as the penis, thus giving the impression of a longer penis. Additionally, owing to the physical makeup of the extension material, the added length will feel natural (i.e. the same as a larger penis) to the user's sexual partner.

Moreover, by only making contact with the extreme tip of the penis, the anterior sheath 14 and extension material 16 of the condom do not affect the sensitivity of the penis, and hence does not reduce the satisfaction of the sexual experience.

FIG. 4 illustrates another embodiment of a condom 10 that further includes the addition of a memory retaining material 17 capable of retaining unique shapes imparted onto it by a user. As described herein, memory retaining material 17 can include any number of synthetic rubber compounds or soft silicone such as Memory Gel®, for example, having a consistency similar to that of putty. Such a feature can allow a user to customize the shape of the anterior section 14 of the condom 10 into any number of different configurations based on the likes and dislikes of the users partner (see arrow A).

Accordingly, an extension condom 10 described herein can provide the look and feel of a larger penis while providing the safety of a traditional disposable condom.

As to a further description of the manner and use of the present invention, the same should be apparent from the above description. Accordingly, no further discussion relating to the manner of usage and operation will be provided.

The terminology used herein is for the purpose of describing particular embodiments only and is not intended to be limiting of the invention. As used herein, the singular forms “a,” “an,” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

The corresponding structures, materials, acts, and equivalents of all means or step plus function elements in the claims below are intended to include any structure, material, or act for performing the function in combination with other claimed elements as specifically claimed. The description of the present invention has been presented for purposes of illustration and description, but is not intended to be exhaustive or limited to the invention in the form disclosed. Many modifications and variations will be apparent to those of ordinary skill in the art without departing from the scope and spirit of the invention. The embodiment was chosen and described in order to best explain the principles of the invention and the practical application, and to enable others of ordinary skill in the art to understand the invention for various embodiments with various modifications as are suited to the particular use contemplated.

What is claimed is:

1. An extension condom comprising:
a first thin elastic tubular sheath configured to receive a penis, said first tubular sheath having an open end and a closed end;
a sperm containment cell integrated into the closed end of the first tubular sheath via a neck;
a second thin elastic tubular sheath connected to the closed end of the first tubular sheath, said second tubular sheath forming a pocket around the sperm containment cell and extending outward along a same plane as the first tubular sheath; and
an extension material positioned within the second tubular sheath, said extension material being configured to provide an appearance and feel of a longer penis, wherein said first and second elastic tubular members are integrally formed so as to conceal the presence of the extension material.

2. The extension condom of claim 1, wherein said condom is disposable.

3. The extension condom of claim 1, wherein said second tubular sheath includes a lateral dimension between ¼ inch and two inches.

4. The extension condom of claim 1, wherein said extension material includes at least one of soft rubber, foam, liquid, gel and formed latex.

5. The extension condom of claim 1, wherein each of the first and second tubular members includes at least one of latex, polyurethane and lamb skin.

6. The extension condom of claim 1, wherein each of the first and second tubular members includes at least one of an artificial color and marking.

7. An extension condom comprising:
a first thin elastic tubular sheath configured to receive a penis, said first tubular sheath having an open end and a closed end;
a sperm containment cell integrated into the closed end of the first tubular sheath via a neck;
a second thin elastic tubular sheath connected to the closed end of the first tubular sheath, said second tubular sheath forming a pocket around the sperm containment cell and extending outward along a same plane as the first tubular sheath; and
an extension material positioned within the second tubular sheath, said extension material including a memory material configured to be shaped by a user, wherein said first and second elastic tubular members are integrally formed so as to conceal the presence of the extension material.

8. The extension condom of claim 7, wherein said condom is disposable.

9. The extension condom of claim 7, wherein said second tubular sheath includes an adjustable lateral dimension between ¼ inch and two inches.

10. The extension condom of claim 7, wherein each of the first and second tubular members includes at least one of latex, polyurethane and lamb skin.

11. The extension condom of claim 7, wherein each of the first and second tubular members includes at least one of an artificial color and marking.
